(19) United States
(12) Patent Application Publication
(10) Pub. No.: US 2004/0260341 A1
(43) Pub. Date: Dec. 23, 2004

(54) Ophtalmic tissue barrier

(76) Inventor: James C. Hays, Atlanta, GA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/487,794

(22) PCT Filed: Aug. 30, 2002
(86) PCT No.: PCT/GB02/03972

(30) Foreign Application Priority Data
Aug. 31, 2001 (GB) 0121168.9

(51) Int. Cl. A61B 17/08

(52) U.S. Cl. 606/215; 623/4.1; 128/898

(57) ABSTRACT

A tissue barrier (10), for example of titanium alloy, for insertion into an incision in sclera of the eye for the treatment of conditions such as presbyopia, comprises an elongate body which is inert and/or non-reactive in tissue. The barrier can include lateral flanges (14) or not. In a preferred embodiment the barrier has an inverted T-shaped cross-section.

### FIELD OF THE INVENTION

This invention relates to ophthalmic devices and procedures, and is particularly concerned with the treatment of presbyopia.

### BACKGROUND TO THE INVENTION

It is known to make radial incisions extending into the anterior ciliary area of the sclera. Such techniques were used in the early 1980s as part of radial keratotomy procedures. One disadvantage of this procedure was that it could produce a myopic shift. Any procedure which improves the working ability of the ciliary body/zonular complex will enhance accommodation of the eye. Therefore, the use alone of incisions into the sclera will increase the working circumference and enhance accommodation. However, in the healing process, new blood vessels and collagen across the width of the incisions contract as they heal. This results in shrinkage and scarring, and the effect of the original incisions is reduced.

Various attempts have been made to treat presbyopia. In U.S. Pat. No. 5,489,299 the amplitude of accommodation of the eye is increased by manipulating the ciliary muscle through intervention with external means. This can be accomplished by securing a rigid band to the portion of the sclera which radially surrounds the ciliary body.

In U.S. Pat. No. 5,722,952, the sclera is weakened by use of an enzyme which can be injected into the sclera in the region of the ciliary body.

In U.S. Pat. No. 5,354,331 the effective working distance of the ciliary muscle is increased by expanding the sclera in the region of the ciliary body. This is accomplished by suturing to the sclera in the region of the ciliary body a relatively rigid band having a diameter slightly greater than that of the sclera in that region. The scleral expansion band comprises anterior and posterior rims and a web extending between the rims.

### SUMMARY OF THE INVENTION

It is an object of the present invention to enable an effective treatment of presbyopia to be effected, by maintaining the effect of incisions made into the sclera and preventing subsequent shrinkage such as has previously taken place due to the natural healing process.

This is achieved in accordance with the invention by the insertion of tissue barriers into the incisions made in the sclera. A tissue barrier inserted into the sclera will keep the incision open and healing will take place without fibrosis. The tissue barrier will prevent in-growth of tissue. The tissue barriers prevent the contracting which would otherwise occur as part of the healing process, and allow the internal pressure of the eye, i.e. the intraocular pressure, to maintain the stretching which is produced by the original incisions, and thus correcting the presbyopic condition.

It is important to note that the tissue barriers of the present invention are not “implanted” in the sense of being buried in channels created by tunnelling through the sclera. The tissue barriers are placed in the incisions made in the sclera, simply as a barrier to in-growth of new tissue. It is the internal hydrodynamics of the eye, i.e. the constant intraocular pressure, which then effectively stretches the sclera, allowing for increased tension in the ciliary body/zonular complex. The use of the tissue barriers in accordance with the invention creates a passive increase in the intraocular pressure.

The tissue barriers are placed in four or more incisions in the sclera. The incisions increase the effective working distance of the ciliary muscle, by increasing the radial distance between the crystalline lens and the inner diameter of the ciliary muscle. No “segments” are inserted as none are necessary. The tissue barriers are used simply to maintain the integrity of the incisions and allow continued stretching by internal ocular pressure. There is no manipulation of the ciliary muscle involved in the procedures according to the present invention. The tissue barriers serve only to maintain the integrity of the incisions.

In accordance with one aspect of the present invention there is provided a tissue barrier comprising an elongate body which is inert and/or non-reactive in tissue and adapted for insertion into an incision in the sclera of the eye to prevent tissue in-growth.

In a preferred embodiment, the elongate body is provided with lateral flanges to prevent the barrier from being ejected from the incision.

Preferably, the tissue barrier comprises an inverted T-shaped cross-section body with the head of the “T” providing the lateral flanges.

Although the tissue barriers can be of any material which is inert and/or non-reactive in tissue, barriers of titanium or a titanium alloy are particularly preferred.

Also in accordance with the present invention there is provided a method of treating ophthalmic conditions, such as presbyopia, which comprises making a plurality of radial incisions in the sclera of the eye, and inserting an elongate tissue barrier which is inert and/or non-reactive in tissue into each incision to prevent tissue in-growth.

Preferably, after making each incision, lateral extensions of the incision are made at the base of each incision, to receive flange portions of the elongate barrier.

Preferably, an inverted T-shaped cross-section incision is made in the sclera so that the flanges of the barrier are held in place.

### BRIEF DESCRIPTION OF THE DRAWINGS

In order that the invention may be more fully understood, a number of presently preferred embodiments of tissue barrier in accordance with the invention will now be described by way of example and with reference to the accompanying drawings. In the drawings:

FIG. 1 shows a simple tissue barrier in accordance with the invention;

FIG. 2 is a top plan view of a second embodiment of tissue barrier in accordance with the invention;

FIG. 3 is a side view of the tissue barrier of FIG. 2;

FIG. 4 is an end view of the tissue barrier of FIGS. 2 and 3;

FIG. 5 is a top plan view of a third embodiment of tissue barrier in accordance with the invention; and

FIG. 6 is a top plan view of a fourth embodiment of tissue barrier in accordance with the invention.

### DESCRIPTION OF THE PREFERRED EMBODIMENTS

The tissue barriers shown in the drawings are preferably made of a titanium alloy, although other materials which are inert and/or non-reactive in tissue could be used.

In the drawings all the indicated dimensions are given in millimetres (mm).

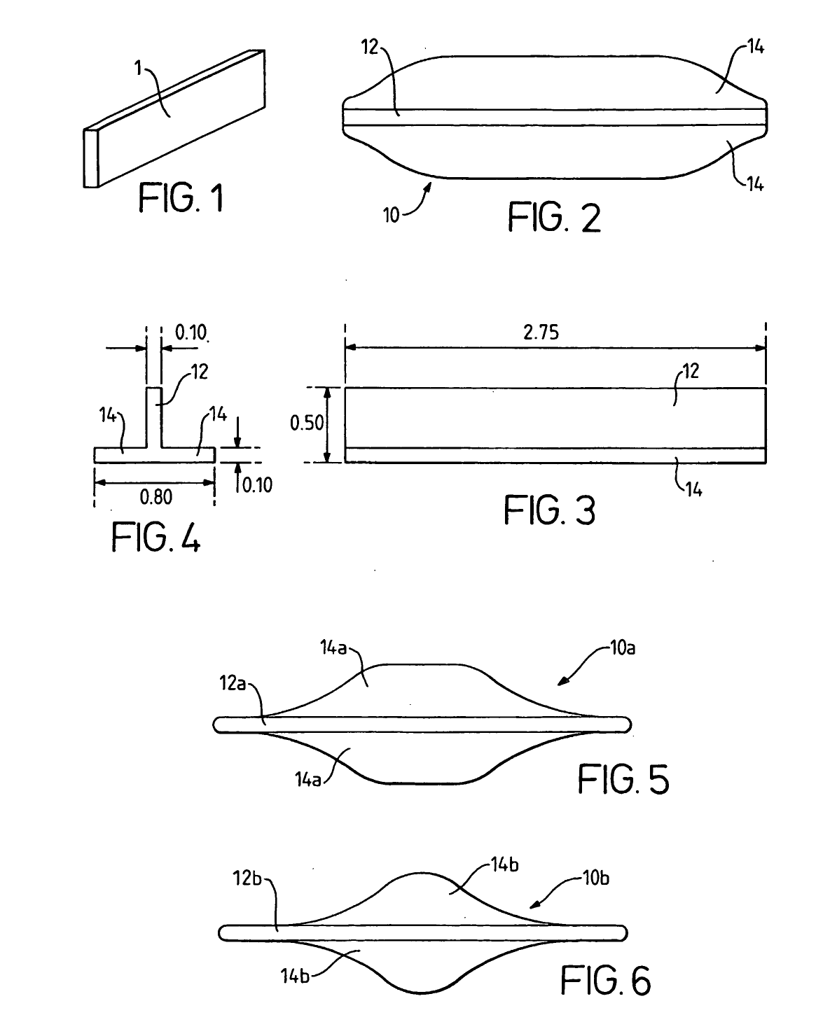

Referring first to FIG. 1, this shows a tissue barrier in the form of an elongate fillet 1 of titanium or titanium alloy or other material which is inert and/or non-reactive in tissue. The fillet is parallelepiped in shape and may have slightly rounded edges and/or corners.

Referring now to FIGS. 2 to 4, there is shown a tissue barrier 10 which is of inverted T-shaped cross-section, as shown most clearly in FIG. 4. The tissue barrier comprises a central longitudinal stem 12 with the head of the T forming lateral flanges 14 on each side of the stem. In this embodiment the flanges 14 extend substantially the full length of the barrier, being tapered towards the ends. In a practical embodiment the tissue barrier is 2.75 mm in length, 0.80 mm in width, and with the stem 12 having a thickness of 0.10 mm. The depth of the barrier is 0.50 mm. An advantage of the use of flanges 14 of substantial length is that this minimises any tendency of the tissue barrier to “rock” within the incision. To accommodate a tissue barrier of these dimensions, each incision in the sclera is 3.5 mm long and 0.60 mm deep. The initial radial incision in the sclera is followed by the use of a dissector which can be rotated to make the lateral incisions which result in a suitable inverted T-shaped cross-section incision which can receive the tissue barrier. Four or more radial incisions are made in the sclera and a tissue barrier is inserted in each incision.

A third embodiment of tissue barrier 10a is shown in FIG. 5. Here, the transverse flanges 14a are shorter in length than in the second embodiment and have a curved outline. The overall dimensions are the same as for the second embodiment. FIG. 6 shows a fourth embodiment of tissue barrier 10b in accordance with the invention, where the flanges 14b are of further reduced length and are concentrated towards the centre of the longitudinally extending stem 12b. This results in a more rounded flat configuration for each of the transverse flanges 14b.

Other configurations of transverse flange can be used and the tissue barrier can have dimensions other than those specified above.

It is to be understood that even though numerous characteristics and advantages of the present invention have been set forth in the foregoing description, together with details of the structure and function of the invention, these are given by way of example only and changes may be made, particularly in matters of size and shape, within the spirit and scope of the appended claims.

1. A tissue barrier comprising an elongate body which is non-reactive in tissue and adapted for insertion into an incision in a sclera of an eye to prevent tissue in-growth.

2. A tissue barrier as claimed in claim 1, in which the elongate body is parallelepiped in shape.

3. A tissue barrier as claimed in claim 1, in which the elongate body comprises a longitudinally extending stem portion provided with lateral flanges to prevent the barrier from being ejected from the incision.

4. A tissue barrier as claimed in claim 3, in which the elongate body has a cross-section shaped like an inverted T having a head, with the head of the T providing the flanges.

5. A tissue barrier as claimed in claim 3, in which the lateral flanges extend substantially the full length of the elongate body.

6. A tissue barrier as claimed in claim 3, in which the lateral flanges are in a centre portion only of the elongate body and have a rounded flat shape.

7. A tissue barrier as claimed in claim 3, in which each flange has a thickness about equal to a transverse width of the stem portion.

8. A tissue barrier as claimed in claim 3, in which each flange has a transverse width approximately equal to height of the stem portion.

9. A tissue barrier as claimed in claim 3, in which the barrier has a transverse width of the order of 0.8 mm.

10. A tissue barrier as claimed in claim 3, in which each flange has a thickness of the order of 0.1 mm.

11. A tissue barrier as claimed in claim 3, in which the elongate body has a length of the order of 2.75 mm.

12. A tissue barrier as claimed in claim 3, in which the barrier has a height of the order of 0.5 mm.

13. A method of treating ophthalmic conditions, such as presbyopia, which comprises making a plurality of radial incisions in a sclera of an eye, and inserting an elongate tissue barrier which is non-reactive in tissue into each incision to prevent tissue re-growth.

14. A method as claimed in claim 13, which includes enlarging each radial incision to make incisions of inverted T-shaped cross-section.

15. A method as claimed in claim 13, in which the tissue barrier is a barrier as claimed in claim 1.

16. A method of carrying out ophthalmic surgery comprising utilizing the tissue barrier of claim 1.
